(19) United States
(12) Patent Application Publication
(10) Pub. No.: US 2012/0171663 A1
(43) Pub. Date: Jul. 5, 2012

(54) System for Detection of Occult Murine Cytomegalovirus (MCMV)

(75) Inventor: Charles H. Cook, Bexley, OH (US)

(73) Assignee: THE OHIO STATE UNIVERSITY, Columbus, OH (US)

(21) Appl. No.: 13/395,703

(22) PCT Filed: Sep. 16, 2010
(86) PCT No.: PCT/US10/49100

§ 371 Date: Mar. 13, 2012

Related U.S. Application Data
(60) Provisional application No. 61/243,985, filed on Sep. 18, 2009.
(60) Provisional application No. 61/246,773, filed on Sep. 29, 2009.

(51) Int. Cl. C12Q 1/70; C07H 21/04

(52) U.S. Cl. 435/5; 536/24.33

(57) ABSTRACT

Methods and compositions for detecting, treating, characterizing, and diagnosing cytomegalovirus in mammals using a nested-PCR methodology using specific predesigned primers are described.

### CROSS-REFERENCE TO RELATED APPLICATIONS

This application claims the benefit of U.S. Provisional Application Ser. No. 61/243,985 filed Sep. 18, 2009 and Ser. No. 61/246,773 filed Sep. 29, 2009, the entire disclosures of which are expressly incorporated herein by reference.

### STATEMENT REGARDING FEDERALLY SPONSORED RESEARCH

This invention was made with government support under Grant No. AI053095, awarded by the National Institute of Allergy and Infectious Diseases (NIAID) and Grant No. GM066115, awarded by the National Institute of General Medical Sciences (NIGMS). The government has certain rights in this invention.

### SEQUENCE LISTING

The instant application contains a Sequence Listing which has been submitted via EFS-web and is hereby incorporated by reference in its entirety. The ASCII copy, created on Sep. 10, 2010, is named 604_{—}51122_SEQ_LIST_{—}09035.txt, and is 4,849 bytes in size.

### TECHNICAL FIELD AND INDUSTRIAL APPLICABILITY OF THE INVENTION

The present invention relates to compositions and methods for detecting, treating, characterizing, and diagnosing Herpes related diseases, particularly affecting mammals with compromised immune systems.

### BACKGROUND OF THE INVENTION

Cytomegalovirus (CMV) is a beta-Herpes virus that is readily transmitted from infected to naïve hosts leading to endemic prevalence in humans and wild mice. Wild mice appear to be particularly susceptible, showing nearly 100% prevalence. In immunocompetent hosts, a primary CMV infection does not induce overt symptoms, instead causing a more mild self-limited flu-like illness. After acute infection, the virus is not eradicated, and the infected host often sheds virus in bodily fluids such as saliva or urine, likely contributing to endemic spread. For these reasons, most humans acquire CMV without their direct knowledge, and thus natural infection in mice might go completely unnoticed.

For these reasons, murine cytomegalovirus (MCMV) is routinely screened for by commercial health testing in mice. Interestingly, a recent publication suggests that the prevalence of MCMV in laboratory mice is extremely low (0.04%). Of note, this prevalence is based upon routinely used serologic testing for CMV reactive antibodies, although these tests may be 10-fold less sensitive than PCR.

This lack of sensitivity, however, is a concern in research. For example, transplant research, which utilizes murine models, has been plagued for many years by sporadic, episodic periods during which the normal models of graft acceptance do not work. Despite rather exhaustive efforts, researchers have been unable to determine the cause of these episodes. However, commercial health screenings performed on these and other sentinel mice were negative for infectious pathogens.

It would be useful to have effective methods of screening for MCMV that are more sensitive than currently available screening methods and procedures.

It would also be useful to have methods of screening for MCMV that are capable of providing additional information useful in the treatment of transplant mammalian patients and in the prevention or treatment of allograft rejections in these transplant patients.

### SUMMARY OF THE INVENTION

In one aspect, there are provided an improved diagnostic method for an occult virus in mammalian research animals.

There is also provided herein an improved sensitive method that detects the presence of the MCMV which is an improvement over current routinely used methodologies.

The MCMV diagnostic method and testing system is especially useful for research on mammals with compromised immune systems such as allograft transplant recipients, AIDS and leukemia mammalian models.

Other systems, methods, features, and advantages of the present invention will be or will become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the Patent Office upon request and payment of the necessary fee.

FIG. 1A-FIG. 1B: Development of occult murine cytomegalovirus (MCMV) in vivarium housed mice:

FIG. 1A: Two strains of mice were tested (BALB/c and C57BL-6) upon arrival and after 5 weeks of vivarium-housing. Nested PCR was performed on DNA extracted from salivary glands for MCMV glycoprotein B, and GAPDH was used as a positive control. Upon arrival (day 0) all mice were negative for MCMV DNA. After 5 weeks of vivarium-housing, all mice show MCMV DNA. Neg and Pos refer to negative and positive technique controls. Each lane represents results from single mice.

FIG. 1B: MCMV reactive antibody screening was performed using flow cytometry for vivarium-housed mice. Vivarium-housed mice show MCMV reactive antibody responses comparable to MCMV naïve mice, and significantly lower than experimentally infected mice. Each point represents sera from a single mouse.

FIG. 2. Time-course of occult murine cytomegalovirus (MCMV) infection in vivariumhoused mice. Mice w ere divided into two cohorts after vendor delivery. The isolated group was handled by a single person caring only for this cohort, while the ULAR group was handled per protocol by vivarium staff. Months indicate months of housing before testing by nested PCR for MCMV glycoprotein B (G) DNA and-β-actin (control) in salivary glands. Both cohorts tested negative after month 1. The ULAR group became positive during the second month, and the isolated group tested positive after the third month. Each lane represents results from a single mouse performed in duplicate.

FIG. 3: Wild mice trapped in vivarium harbor murine cytomegalovirus (MCMV). Salivary glands from two wild mice were tested for MCMV DNA using nested PCR. GAPDH was used as a control. Both mice were positive for MCMV DNA Polymerase (DNA Pol), glycoprotein B (GB), and immediate early-1 (IE-1). Neg and Pos refer to negative and positive technique controls. Results were confirmed in triplicate (not shown).

FIG. 4. Mouse chow analysis for murine cytomegalovirus (MCMV) DNA. Mouse chow was evaluated for MCM glycoprotein B (GB) DNA by nested PCR. Chow was obtained from several bins within the vivarium (lanes 1 3), freshly opened chow from two sources (lanes 4 and 5) and chow from a different vivarium (lane 6). Because mouse chow contains maize (corn), maize high mobility gene a (HMG) was used to confirm DNA ex traction. All chow samples tested positive for MCMV GB DNA by nested PCR.

FIG. 5. Murine cytomegalovirus (MCMV) immediate early 1 (IE-1) gene sequence comparison. PCR amplicons for IE-1 (384 bp) w ere sequenced by PCR gel isolation. DNA sequences from chow MCMV (‘Comm Chow’ [SEQ ID NO: 20]), wild MCMV ('Wild 1′ [SEQ ID NO: 17] and ‘Wild 2’ [SEQ ID NO: 18]), and vivarium MCMV [SEQ ID NO: 19] were aligned and compared to lab MCMV [SEQ ID NO: 23] (Smith strain). Shown is a 40 bp sequence that highlights similarities and differences between the MCMV isolates. Dark gray indicates differences unique to wild MCMV, and light gray differences seen in vivarium-acquired MCMV. Sequencing results w ere confirmed in triplicate (not show n). FIG. 5 also discloses ‘Occult 1’, ‘Occult 2’, and ‘Occult 3’ as SEQ ID NOS: 21-23, respectively.

FIG. 6. Immune response to low titer murine cytomegalovirus (MCMV) infection. Cohorts of n=5 BAL B/c mice w ere infected with 10⁰, 10¹, or 10⁶ pfu Smith strain MCMV. Sera were serially obtained by venous puncture 4, 12, and 16 week s after infection, and tissues were obtained 16 weeks after infection. For FIG. 6A and FIG. 6C, points/bars represent mean and standard errors from n=5 mice. For FIG. 6D, each lane represents an individual mouse, and NEG and POS refer to technique controls.

FIG. 6A. MCMV-specific antibody responses to infections were measured by ELISA.

FIG. 6B. Representative flow cytometry scatter plots for MCMV-m164 specific CD8 T-cells from peripheral blood mononuclear cells (PBM).

FIG. 6C. Summary of m164-specific T-cell response to infection from PBMC over time.

FIG. 6D. Nested PCR for MCMV glycoprotein B (GB) from lungs 16 week s after infection.

### DETAILED DESCRIPTION OF THE PREFERRED EMBODIMENT

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

In a broad aspect, there is provided herein a method for detecting murine cytomegalovirus (MCMV) using a set of novel primers for polymerase chain reaction (PCR).

In another broad aspect, there is provided herein a PCR set comprising a primer set including a primer having a nucleotide sequence as set forth in SEQ ID NO: 1, or an operable fragment thereof, and a primer having a nucleotide sequence as set forth in SEQ ID NO:2, or an operable fragment thereof,

wherein the primer set specifically amplifies a target region of murine cytomegalovirus (MCMV) in a polymerase chain reaction (PCR).

In another aspect, there is provided herein a PCR set comprising a nested primer set including a primer having a nucleotide sequence as set forth in SEQ ID NO: 3, or an operable fragment thereof, and a primer having a nucleotide sequence as set forth in SEQ ID NO:4, or an operable fragment thereof,

wherein the nested primer set specifically amplifies a target region of murine cytomegalovirus (MCMV) in a polymerase chain reaction (PCR).

In another aspect, there is provided herein a PCR set comprising a primer set including a primer having a nucleotide sequence as set forth in SEQ ID NO: 1, or an operable fragment thereof, and a primer having a nucleotide sequence as set forth in SEQ ID NO:2, or an operable fragment thereof, and, a nested primer set including a primer having a nucleotide sequence as set forth in SEQ ID NO: 3, or an operable fragment thereof, and a primer having a nucleotide sequence as set forth in SEQ ID NO:4, or an operable fragment thereof;

wherein the nested primer set specifically amplifies a target region of murine cytomegalovirus (MCMV) in a polymerase chain reaction (PCR).

In another aspect, there is provided herein a method for detecting MCMV, which comprises amplifying a nucleic acid sample obtained from a sample by PCR using the PCR sets as described herein.

In certain embodiments, the PCR is a two-step PCR comprising a denaturation step and an annealing and extension step or a three-step PCR comprising a denaturation step, an annealing step, and an extension step.

In certain embodiments, the PCR is performed under conditions of initial denaturation 4 min at 94° C., 35 cycles-denaturation 30 s at 94° C., annealing 30 s at 53° C., elongation 30 s at 72° C., followed by final elongation 7 min at 72° C., then holding at 4° C.

In another aspect, there is provided herein a MCMV detection kit comprising the primer sets described herein.

In another aspect, there is provided herein an isolated nucleic acid comprising a nucleotide sequence as set forth in one of SEQ ID NOs: 1, 2, 3 and 4.

In another aspect, there is provided herein a method of hybridizing the isolated nucleic acid to an isolated cytomegalovirus (CMV) sequence or a CMV sequence derived from an artificial construct, comprising: contacting the nucleic acid to a sample under conditions suitable for hybridization.

In certain embodiments, the method further comprises isolating the hybridized CMV sequence.

In another aspect, there is provided herein a probe that hybridizes to the isolated nucleic acids described herein.

In certain embodiments, the sample comprises one or more of: blood, serum, plasma, sputum, urine, stool, skin, cerebrospinal fluid, saliva, gastric secretions, semen, seminal fluid, breastmilk, tears, oropharyngeal swabs, nasopharyngeal swabs, throat swabs, nasal aspirates, nasal wash, fluids collected from the ear, eye, mouth, respiratory airways, spinal tissue or fluid, cerebral fluid, trigeminal ganglion sample, a sacral ganglion sample, adipose tissue, lymphoid tissue, placental tissue, upper reproductive tract tissue, gastrointestinal tract tissue, male genital tissue and fetal central nervous system tissue.

In certain embodiments, the sample is from a mouse. In certain embodiments, the sample is from a human.

In another aspect, there is provided herein a composition comprising a substrate attached to at least one detectable moiety, the substrate comprising at least one nucleic acid sequence as described herein.

In another aspect, there is provided herein a method for detecting at least one virus in a sample, the method comprising:

a) contacting the sample with at least one of the compositions described herein, under conditions allowing cleavage of the substrate; and

b) monitoring cleavage of the substrate, wherein the cleavage of the substrate is indicative of the presence of the at least one virus in the sample.

In another aspect, there is provided herein a diagnostic kit for detection of at least one virus in a sample, the kit comprising at least one composition, and reagents for detecting cleavage of the substrate.

In another aspect, there is provided herein use of the nucleic acids described herein for the manufacture of a medicament identified for treating viral infection.

In another aspect, there is provided herein an oligonucleotide primer comprising the sequence (SEQ ID NO:1, 2, 3 or 4) or a portion of SEQ ID NO: 1, 2, 3 or 4,

wherein the primer comprises at least one unique nucleotide as identified as a mismatch between strains of murine cytomegalovirus (MCMV) from other herpes viruses, and distinguishes from other strains of herpes virus.

In another aspect, there is provided herein a kit for detecting murine cytomegalovirus (MCMV), comprising: (a) a pair of flanking primers consisting of SEQ ID NO:1 and SEQ ID NO:2 and (b) a pair of nested primers consisting of SEQ ID NO:3 and SEQ ID NO: 4.

In another aspect, there is provided herein a method for detecting murine cytomegalovirus (MCMV) by using the kit, comprising:

a) providing an amplified cDNA of a sample though RT-PCR by a pair of primers consisting of SEQ ID NO:1 and SEQ ID NO:2;

b) discriminating the amplified cDNA of step (a) though nested PCR by a pair of SNP primers consisting of SEQ ID NO:3 and SEQ ID NO:4; and

c) identifying the amplified cDNA of step (b).

In another aspect, there is provided herein a method for differentiating an infectious strain from a vaccine strain by using the kit, comprising:

a) providing an amplified cDNA of a sample through RT-PCR by a pair of primers having a nucleotide sequence as set forth in SEQ ID NO:1 and SEQ ID NO:2, wherein the pair of primers share the same template from the infectious and vaccine strains;

b) discriminating the amplified cDNA of step (a) through nested PCR by a pair of nested primers having a nucleotide sequence as set forth in SEQ ID NO:3 and SEQ ID NO:4, which creates incomplete complementarity to the amplified cDNA of step (a); and

c) identifying the size of the amplified cDNA of step (b).

In another broad aspect, there is provided herein a method of detecting and identifying mammalian Herpesvirus of the type Cyclomegalovirus in a sample. The method includes:

(a) applying onto a supporting substrate consensus DNA polymerase gene standards corresponding to respective mammalian Cyclomegalovirus;

(b) isolating DNA from the mammalian sample;

(c) amplifying the isolated DNA by a round of PCR using designed flanking primers of the sequences:

[SEQ ID NO: 1]

| | |
|---|---|
| Flanking Primer-left: | ctg ggc gag aac aac gag at; |
| and | |

[SEQ ID NO: 2]

| | |
|---|---|
| Flanking Primer-right: | cgc agc tct ccc ttc gag ta,, |

thereby generating PCR products;

(d) hybridizing the PCR products to the supporting substrate; and

e) detecting bound PCR products via:
i) the supporting substrate using electrophoresis wherein the presence of bound PCR product to a consensus DNA standard for a mammalian Herpesvirus indicates the presence of the mammalian Herpesvirus in the sample, or
ii) separating the amplified sequencing products to obtain nucleic acid sequence data; and analyzing the nucleic acid sequence data on comparison with a Cytomegalovirus consensus sequence.

In certain embodiments, the PCR product is subject to a second PCR cycle, comprising:

(a) applying onto a supporting substrate consensus DNA polymerase gene standards corresponding to respective mammalian Cyclomegalovirus;

(b) isolating DNA of first round PCR;

(c) amplifying the isolated DNA by a round of PCR using designed nested primers:

[SEQ ID NO: 3]

| | |
|---|---|
| Nested Primer-left: | gag aac tgc gac acg aac ag; |
| and | |

[SEQ ID NO: 4]

| | |
|---|---|
| Nested Primer-right: | agc acc ttg aag tcg gtg tt, |

thereby generating PCR products;

(d) hybridizing the PCR products to the supporting substrate; and

(e) detecting bound PCR products via:
i) the supporting substrate using electrophoresis wherein the presence of bound PCR product to a consensus DNA standard for a mammalian Herpesvirus indicates the presence of the mammalian Herpesvirus in the sample, or
ii) separating the amplified sequencing products to obtain nucleic acid sequence data; and analyzing the nucleic acid sequence data on comparison with a Cytomegalovirus consensus sequence.

In certain embodiments, the sample comprises one or more of: saliva, saliva gland extract, urine, bladder extract, kidney extract, blood, lung extract, tears, semen or breast milk. Non-limiting examples of mammals include: mouse, rat, guinea pig, swine, pig, hog or boar. In particular embodiments, the mammal can be a mammal used for research. Non-limiting examples include immunocompromised mammals where the immunocompromised mammals are comprised of animals receiving allograft; mammals infected with AIDS or AIDS like virus; mammals having leukemia; mammal fetus; newborn mammals.

In another broad aspect, there is provided herein a kit useful for detecting and identifying mammalian Herpesvirus in a sample where the kit includes: i) consensus DNA polymerase gene standards corresponding to mammalian Cytomegalovirus; ii) a supporting substrate to which the consensus DNA standards can be attached; iii) PCR primers: Flanking Primer—left: ctg ggc gag aac aac gag at [SEQ ID NO:1]; and iv) Flanking Primer—right: cgc agc tct ccc ttc gag ta, [SEQ ID NO:2].

In certain embodiments; the kit contains materials for a second stage in which the PCR product is subject to a second PCR cycle, comprising: Nested Primer—left: gag aac tgc gac acg aac ag [SEQ ID NO:3]; and Nested Primer—right: agc acc ttg aag tcg gtg tt [SEQ ID NO:4].

In another broad aspect, there is provided herein a method of avoiding Herpes related complication in surgical procedure on mammals, prior to surgery, by detecting and identifying mammalian Herpesvirus of the type cytomegalovirus in a sample using the methods described herein. In certain embodiments, the surgery is tissue transplantation, including but not limited to, allografts, isografts or xenografts. Non-limiting examples include where the surgery is on an immunocompromised patient including but not limited to, mammals infected with AIDS or AIDS like virus; mammals having leukemia; mammal fetus; newborn mammals.

In another broad aspect, there is provided herein a method for identifying mammalian Herpesvirus infected cells in a cell culture or sample which uses the method described herein. In certain embodiments, the sample comprises a cell culture medium containing mammalian cultured cells and/or harvested from mammals, such as, but not limited to embryonic cells.

The method described herein provides a sensitive methodology that has now been shown to be about a 10 fold times more sensitive than current routinely used methods.

The method described herein also is useful with readily available equipment and can be employed across a variety of known PCR platforms.

The method described herein also provides a robust, high fidelity method for the detection of MCMV.

The method is especially useful for screening research mice and for detecting MCMV which can negatively influence allograft acceptance in cardiac transplantation mouse models.

The present invention is based, at least in part, on the inventor's discovery of an association between occult MCMV and unexpected allograft rejections.

The method described herein has now been used to show that vivarium-housed “naïve” mice can acquire MCMV within 30-60 days after arrival.

In one particular aspect, in order to detect the MCMV virus, the inventor herein has developed polymerase chain reaction (PCR) primers that are used in a highly sensitive nested-PCR reaction.

In one embodiment, the method includes the use of newly designed sets of flanking primers and nested primers. In the nested-PCR methodology, two successive rounds of PCR are performed, thus reducing background due to non-specific amplification of DNA. The first set of primers includes the flanking primers, and the second set includes the nested primers.

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference. The following examples are intended to illustrate certain preferred embodiments of the invention and should not be interpreted to limit the scope of the invention as defined in the claims, unless so specified.

The value of the present invention can thus be seen by reference to the Examples herein.

### EXAMPLES

In transplantation tolerance studies in laboratory mice, there have been intermittent periods of unexplained allograft failures in control mice. Because these failures were episodic and often involved entire cohorts within a single cage, the inventor herein suspected an infectious etiology, yet commercial serologic evaluations of these and other sentinel mice were routinely negative for infectious pathogens (unpublished data). Murine cytomegalovirus (MCMV) can influence cardiac allograft acceptance. Further, vivarium-housed “naïve” mice can acquire MCMV. The inventor herein believed that the colonies might intermittently be experiencing occult MCMV infections.

CMV is a beta-herpesvirus that is readily transmitted from infected to naive hosts leading to endemic prevalence in both humans and wild mice. In immunocompetent hosts, primary CMV infection does not usually induce overt pathology, instead causing a self-limited flu-like illness. After acute infection the virus is not eradicated, and the infected host often sheds virus in saliva or urine, likely contributing to endemic spread. In mice, natural infections occur without clinical signs and thus without careful screening MCMV could go completely unnoticed.

MCMV is routinely included in commercially available murine health screening panels, although a recent publication suggests that the prevalence of MCMV in laboratory mice is extremely low (0.04%). This is in contrast to near 100% prevalence in wild mice. Of note, laboratory mouse prevalence is based upon serologic testing for MCMV-reactive antibodies. Despite these very low reported rates of MCMV in vivarium-housed mice, the inventor's laboratory experience with highly sensitive PCR techniques led the inventor to hypothesize that vivarium-housed mice might acquire MCMV that escapes commercially available serologic detection, a condition the inventor herein terms “occult” infection, and that occult MCMV infection might influence transplant allograft acceptance.

Materials and Methods

Animals

Female BAL B/c and C57 BL/6 mice (Harlan, Indianapolis Ind.) 6-8 weeks of age were used. All animals were housed in an AAALAC-accredited animal facility, adhering to the Guide for the Care and Use of Laboratory Animals prepared by the National Research Council (NIH Publication No. 86-23, revised 1996) with approval of the Ohio State University Institutional Animal Care and Use Committee. All mice were housed in individually ventilated, autoclaved micro-isolator cages with automatic reverse osmosis water supply. Teklad corncob bedding and irradiated 7912 chow ad lib (Harlan, Indianapolis Ind.). Personnel were required to wear full barrier protection in the animal room, with all mouse manipulations done within a biosafety cabinet. SporKlenz disinfectant was used on all surfaces within the biosafety cabinet, as well as cages after use. Mice were euthanized by cervical dislocation under isourane inhalation anesthesia. Mouse tissues were dissected aseptically and frozen immediately in liquid nitrogen, then stored at 80° C. Tissues were procured similarly after euthanasia for wild mice, which were trapped in the vivarium building as part of this facilities routine pest control.

Virus

Purified Smith strain (VR-1399) murine CMV obtained from ATCC (Rockville, Md.) was used for positive controls for both PCR and sequencing. Occult/wild MCMV virus recovery was attempted by murine fibroblast culture with centrifugal enhancement of infectivity using multiple tissues from study mice including lung, spleen, salivary gland, kidney, and bladder. For deliberate mouse infections, mice received 1, 10, or 10⁶ pfu Smith MCMV via intraperitoneal injection.

PCR Primers:

Sequences for murine CMV glycoprotein B (GB), Immediate Early 1 (IE1) and DNA Polymerase (DP) genes were obtained from GenBank (NCBI). The following primers were used for all CMV PCR and RT-PCR reactions.

| Flanking Primers: | |
|---|---|
| [SEQ ID NO: 1] | |
| LEFT PRIMER | ctg ggc gag aac aac gag at |
| [SEQ ID NO: 2] | |
| RIGHT PRIMER | cgc agc tct ccc ttc gag ta |
| Nested Primers: | |
| [SEQ ID NO: 3] | |
| LEFT PRIMER | gag aac tgc gac acg aac ag |
| [SEQ ID NO: 4] | |
| RIGHT PRIMER | agc acc ttg aag tcg gtg tt |
| Primers: | |
| [SEQ ID NO: 5] | |
| IE1 3′ | tag cca atg ata tct tcg agc g |
| [SEQ ID NO: 6] | |
| IE1 5′ | atc tgg tgc tcc tca gat cag cta a |
| (603 bp product). | |
| [SEQ ID NO: 7] | |
| DP 3′ | ggg acc cta ctc cga cga cgt g |
| [SEQ ID NO: 8] | |
| DP 5′ | gct ctg ctc ttc gat cgg tag g |
| (586 bp product). | |
| CMV Nested Primers: | |
| [SEQ ID NO: 9] | |
| IE1 3′ | aca acg caa gat gat ata cgg c, |
| [SEQ ID NO: 10] | |
| IE1 5′ | act acc aca tgt gtg gat acg ct |
| (371 bp product). | |
| [SEQ ID NO: 11] | |
| DP 3′ | gac ctc aag aac acc gtc tac g |
| [SEQ ID NO: 12] | |
| DP 5′ | cag gaa gcg cgc caa ggg atc |

(224 bp product).

Primers for maize high mobility gene a (HMGa) were:

| [SEQ ID NO: 13] | |
|---|---|
| forward | TGG ATT CCA TCA ATG CAA AA, |
| [SEQ ID NO: 14] | |
| reverse | GAG GAG CTC CAT CAC TCG TC. |

Primers for β-actin, glyceraldehyde 3-phosphate dehydrogenase (GAPDH), and MCMV viral DNA polymerase (D Pol) were also used

PCR

DNA were extracted from tissues homogenates using DNeasy Blood & Tissue Kit (QIAGEN GmbH, Hilden, Germany). DNA were eluted in 100 μl of distilled water and stored at −20° C. until analysis. DNA were amplified in a total volume of 25 μl with 200 nM of each primer and 1.0 U of Taq DNA polymerase (GIBCO BRL) added in 2.5 μl of a PCR buffer (50 mM KCl, 20 mM Tris-HCl (pH 8.4), and 1.5 mM MgCl₂). PCR reactions were carried out using a Perkin Elmer 9700 thermocycler (PE Applied Biosystems, Foster City, Calif.), using the following program: initial denaturation 4 min at 94° C., 35 cycles-denaturation 30 s at 94° C., annealing 30 s at 53° C., elongation 30 s at 72° C., followed by final elongation 7 min at 72° C., then hold at 4° C.

Primers used for transcription of IE1, GB and DNA polymerase genes are listed above. Beta-actin or GAPDH transcripts were used as a cellular transcript control. Amplification products were separated by electrophoresis in 1% agarose gels, and gels were stained with ethidium bromide.

DNA Sequencing and Analysis:

PCR products were extracted using QIAquick Gel Extraction Kit (Qiagen). The product was eluted in 50 μl of extraction buffer, and 10 μl of this product was mixed with 2 μl of the appropriate PCR primer. These samples were analyzed using BigDye Terminator Reaction Chemistry v3.1 for sequence analysis on an Applied Biosystems 3730 DNA Analyzer. Further analyses of the sequences were performed using Bioedit Sequence Alignment Editor v7.0.9.0 (Ibis Biosciences, Carlsbad Calif.). Sequences obtained were compared to MCMV strain sequences referenced in Genbank and to sequenced laboratory strains of MCMV.

DNA were extracted from tissue homogenates using DNeasy Blood & Tissue Kit (QIAGEN GmbH, Hilden, Germany). DNA were extracted from chow using Quick Extract Plant DNA extraction solution (EPICENTRE Biotechnologies, Madison Wis.) yielding concentrations of −200 ng/1. DNA were eluted in 100 μl of distilled water and stored at 20° C. until analysis. DNA were amplified in a total volume of 25 μl with 200 nM of each primer and 1.0 U of Taq DNA polymerase (GIBCO BRL) added in 2.5 μl of a PCR buffer (50 mM KCl, 20 mM TrisHCl (pH 8.4), and 1.5 mM MgCl₂). PCR reactions were carried out using a Perkin Elmer 9700 thermocycler (PE Applied Biosystems, Foster City, Calif.), using the following program: initial denaturation 4 min at 94° C., 35 cycle-denaturation 30 s at 94° C., annealing 30 s at 53° C., elongation 30 s at 72° C., followed by final elongation 7 min at 72° C., then hold at 4° C. Beta-actin or GAPDH transcripts served as cellular transcript controls. Amplification products were separated by electrophoresis in 1% agarose gels, and gels were stained with ethidium bromide.

Antibody Detection

Sera were evaluated by Charles River Research Animal Diagnostic Services (Wilmington, Mass.) for MCMV-reactive antibody by enzyme-linked immunosorbent assay ELISA.

DNA Sequencing and Analysis

PCR products were extracted using QIAquick Gel Extraction Kit (Qiagen). Products were eluted in 50 μl of ex traction buffer, and 10 μl of this product was mixed with 2 μl of the appropriate PCR primer. These samples were analyzed using BigDye Terminator Reaction Chemistry v3.1 for sequence analysis on an Applied Biosystems 37 30 DNA Analyzer. Further analyses of the sequences were performed using Bioedit Sequence Alignment Editor v7.0.9.0 (Ibis Biosciences, Carlsbad Calif.). Sequences obtained were compared to MCMV strain sequences referenced in GenBank and to sequenced laboratory strains of MCMV.

Cardiac Allograft Tissues

H2^{d} cardiac allografts are heterotopically transplanted into H2^{b} recipients and long term acceptance of these fully mismatched grafts is induced by gallium nitrate. Impulses of these grafts are monitored after transplantation, and at studies conclusion grafts are procured aseptically and frozen. For the current example, the inventor utilized historical tissues from mice with unexplained cardiac allograft failures (n=33, impulses of <2), and mice with successful graft acceptance (n=26, impulses ≧2). Cardiac allografts from these mice were evaluated by nested PCR for the presence of MCMV DNA.

Antibodies and Flow Cytometry

MCMV-specific T-cells were identified using MHC-I tetramers specific for MCMV proteins pp 89 (H2L^{d}-restricted¹⁶⁸YPHFMPTNL¹⁷⁶) [SEQ ID NO:15] and m164 (H2D^{d}-restricted ²⁵⁷AGPPRYSRI²⁶⁵ [SEQ ID NO:16]).

Briefly, blood was collected via submandibular puncture into 30 pl of ACD solution. RBCs were lysed with ACK Lyses buffer (Biowhitaker) and peripheral blood mononuclear cells (PBMC) were divided and washed with FACs buffer. MHC class I peptide tetrameric complexes were produced and assembled. PBMC were incubated with tetramers (37° C.) for 1 h followed by antibody surface staining (4° C.) with fluorescent dye-conjugated antibody specific for CD8 for 1 h (Percy) (BD Harlingen, San Diego, Calif.). Cells were fixed and analyzed by flow cytometry (FACScalibur, Becton Dickinson, Mountain View, Calif.). Lymphocytes were gated by forward-side scatter and 5×10⁵ events acquired for each specimen. Results were analyzed using FlowJo software (Tree Star Inc., Ashland, Oreg.).

Results

Vivarium-Housed Mice can Develop Occult CMV

The data show that mice housed in the vivarium can acquire occult MCMV infections. It was hypothesized that a) mice arrive from vendors already infected, or b) they acquire MCMV after delivery. To test these hypotheses, mice underwent serologic testing for MCMV by Charles River Laboratories as w ell as in-house salivary gland analysis for MCMV DNA. Submandibular salivary glands were chosen to evaluate for viral DNA because they are a major reservoir of MCMV. These evaluations were performed immediately upon arrival and after 5 weeks of vivarium housing. Both MCMV susceptible “(BALB/c) and MCMV “resistant” (C57BL/6) mouse strains were evaluated.

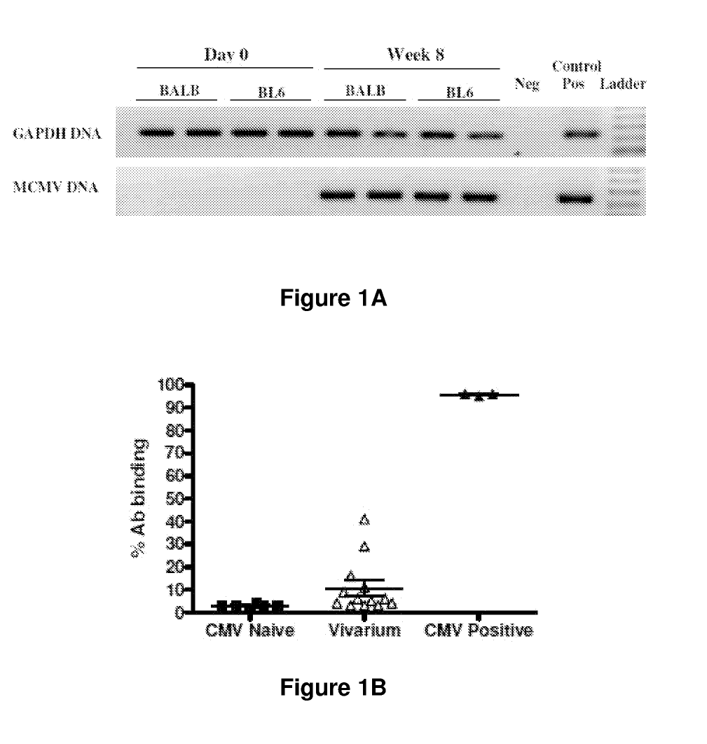

As shown in FIG. 1, mice arriving from the vendor were negative for MCMV DNA by PCR. In contrast, both susceptible and resistant mice housed in the vivarium for 5 weeks became MCMV DNA positive. Concomitantly performed commercial serologic antibody testing was negative for murine pathogens including MCMV (data not shown). This confirmed that mice housed in the barrier vivarium can develop occult MCMV infection that can be missed by commercially available serologic testing.

Timing of MCMV Infection In Vivarium

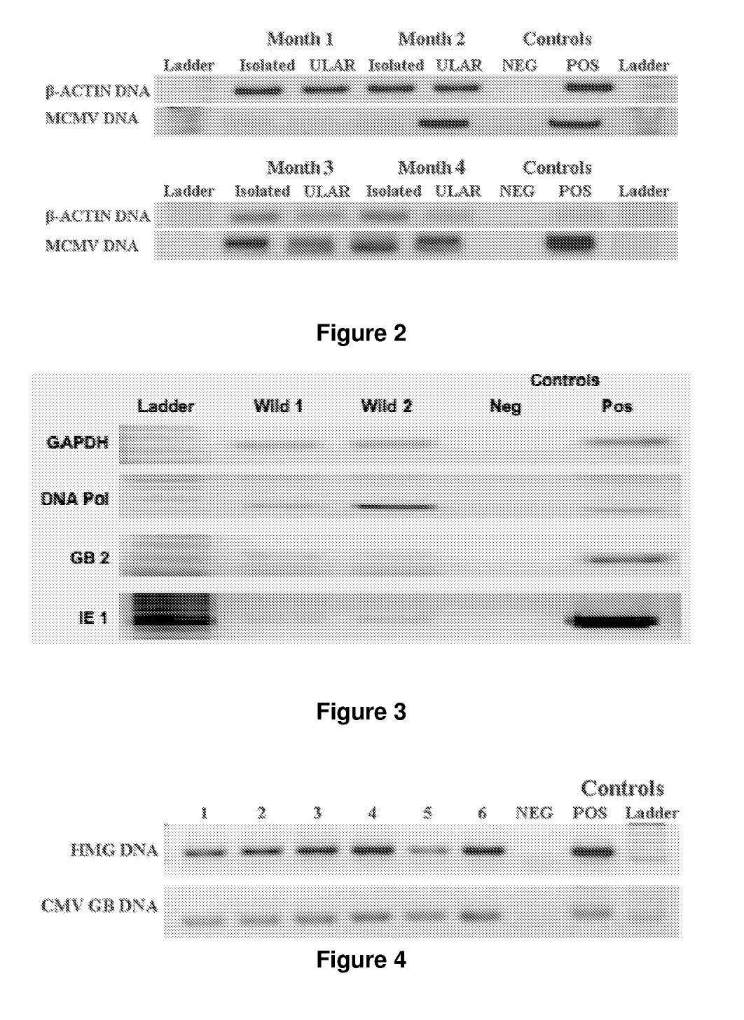

Tests were conducted to determine whether viral transmission might be a consequence of animal handling. Therefore, two additional cohorts of mice were studies to determine the timing of occult MCMV infection in the inventor's facility. One cohort was managed by a single person whose sole vivarium responsibility was caring for this cohort. The second cohort was managed by vivarium staff responsible for the remaining mouse colonies. As shown in FIG. 2, isolated care seemed to delay development of MCMV somewhat, but it did not prevent eventual viral transmission. All mice were serologically negative for MCMV antibody by commercial testing. This showed that vivarium-housed mice can develop occult MCMV infection after 30-60 days of barrier housing even with dedicated handling.

Sequence Confirmation of MCMV

Efforts to recover live virus from vivarium-housed mice in fibroblast cultures after spin inoculation were unsuccessful using salivary gland, spleen, lungs, kidneys, or bladder (not shown). This prompted some concern that this occult virus might not be MCMV, but some similar or previously uncharacterized herpes-family virus. To confirm, MCMV genes GB and IE-1 were cloned and sequenced from mice with occult infection. GG and IE-1 genes were chosen because both are highly conserved in laboratory and wild MCMV strains. For a comparator, these gene regions were cloned and sequenced from Smith strain MCMV, which were identical to those reported in GenBank (100% similarity). Sequences from vivarium-housed mice with occult infection were aligned and compared to Smith MCMV. Occult virus sequences had high similarity to Smith MCMV for both GB (98.2% of 236 bp) and IE (99.1% of 384 bp) genes. This shows that the occult infection observed in vivarium-housed mice is indeed MCMV.

Vehicle of Transmission

During these studies two wild mice were captured by vivarium staff familiar with the experiment. These appeared to be wild and not escaped vivarium mice based on their smaller size and brown coat color.

Nested PCR performed on salivary gland DNA revealed that these wild mice harbored MCMV (FIG. 3).

These wild mice were thus a potential vehicle of MCMV transmission, but because ventilated micro-isolator barrier housing made direct contact impossible, another source was investigated. One common point of contact might be food, and that wild mice could be contaminating vivarium chow that was subsequently distributed to the colony. Mouse chow from several locations within the vivarium was tested for MCMV DNA by nested PCR. Negative controls included chow from another vivarium, and unopened bags from the vivarium and a local vendor. Teklad 7912 chow contains significant maize (corn) substrate, so we amplified maize HMGa DNA for DNA controls. All chow tested positive for MCMV DNA (FIG. 4), despite the source (potentially contaminated versus new).

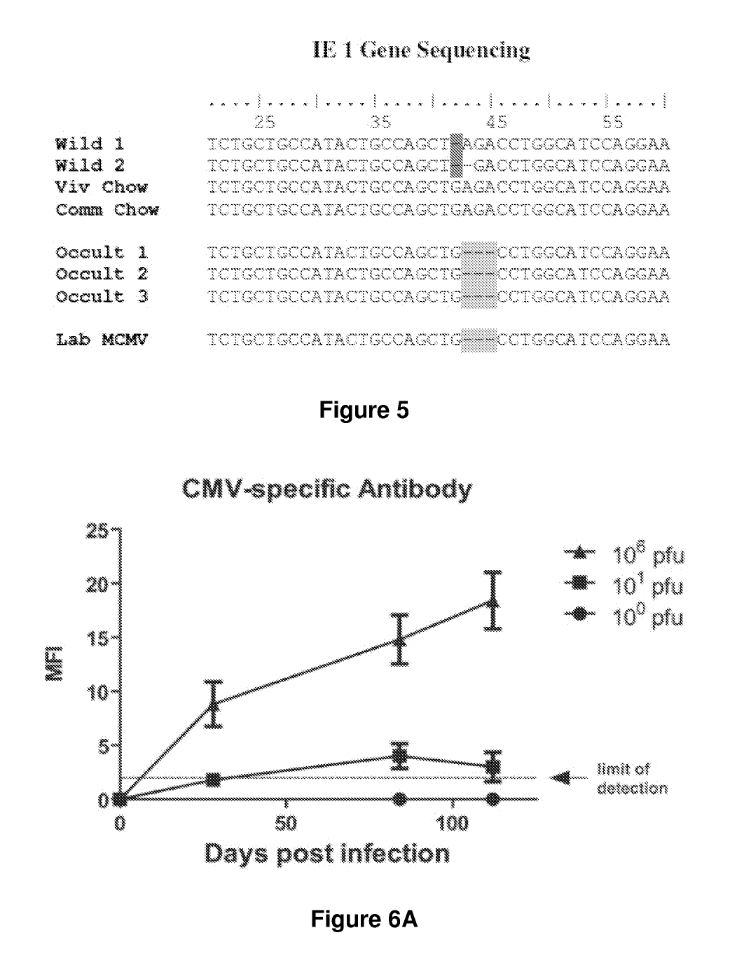

To test whether mouse chow might be the viral reservoir, PCR products for MCMV IE—1 (38 4 bp) from chow were sequenced and compared to sequences from vivarium-housed and wild mice. For illustrative purposes, a 40 bp segment is shown in FIG. 5, showing that wild mice and chow MCMV sequences had high similarity, but that both show important differences from occult MCMV isolates (showing Wild 1 [SEQ ID NO:17], Wild 2 [SEQ ID NO:18], Vivarium Chow SEQ ID NO:19], Commercial Chow[SEQ ID NO:20], Occult 1 [SEQ ID NO:21], Occult 2 [SEQ ID NO:22], Occult 2 [SEQ ID NO:23], and Lab MCMV [SEQ ID NO:23].

Interestingly, vivarium mice with occult MCMV had sequences similar to laboratory MCM. Because wild MCMV are known to have sequence variations when compared with lab strains, and the sequences for wild mice and chow are similar to each other and distinct from vivarium-housed mice with occult infection, the inventor concluded that occult MCMV is not being transmitted to vivarium-housed mice either directly by wild mice or indirectly via chow.

MCMV in Transplant Recipients with Unexpected Allograft Rejection

To determine if the presence of occult MCMV has experimental relevance, the inventor studied cardiac allograft acceptors and unexpected rejectors for evidence of occult MCMV. Frozen cardiac allografts from supposed MCMV-naive mice from previous experiments were evaluated by nested PCR for the presence of MCMV DNA. As shown in Table 1, twenty mice had occult MCMV detected in their allograft hearts, and mice with occult MCMV were twice as likely to experience graft failure than mice without occult MCMV (85% versus 41%, Fishers Exact Test, p=0.002). That is, of mice with unexplained graft failures, 52% had occult MCMV, compared with only 12% of mice with allograft acceptance. All tissues required nested PCR to detect MCMV DNA, and it was impossible to quantitate viral load in these tissues (not shown).

TABLE 1
Murine cytomegalovirus (MCMV) status

DNA Status

| | MCMV+ | MCMV− |
|---|---|---|
| Failed | 17 | 16 |
| Accepted | 3 | 23 |

MCMV-Specific Antibody and T-Cell Responses after Low Titer Infection

These results show that occult MCMV infections are very low titer infection and do not induce significant antibody responses. To test, mice were infected with very low doses of MCMV (10⁰ and 10¹ pfu) and measured MCMV-specific antibody responses over time.

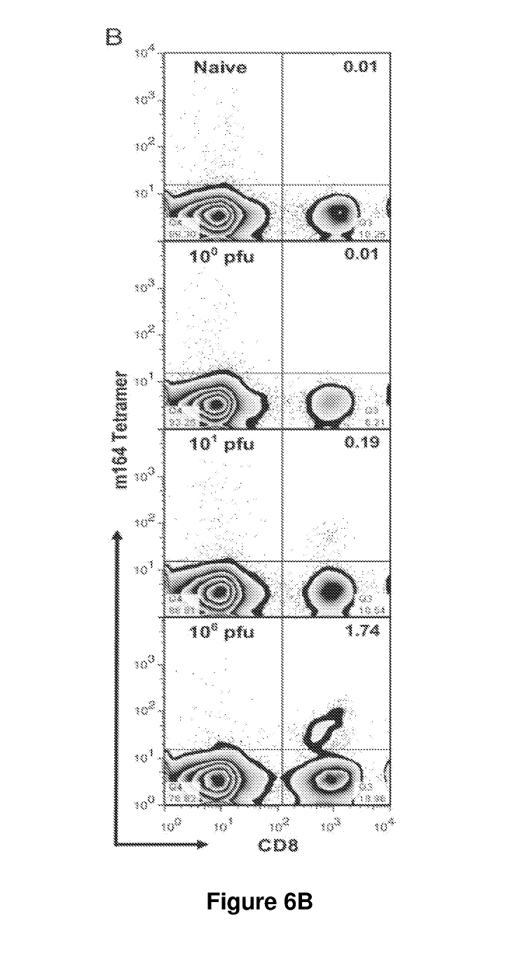

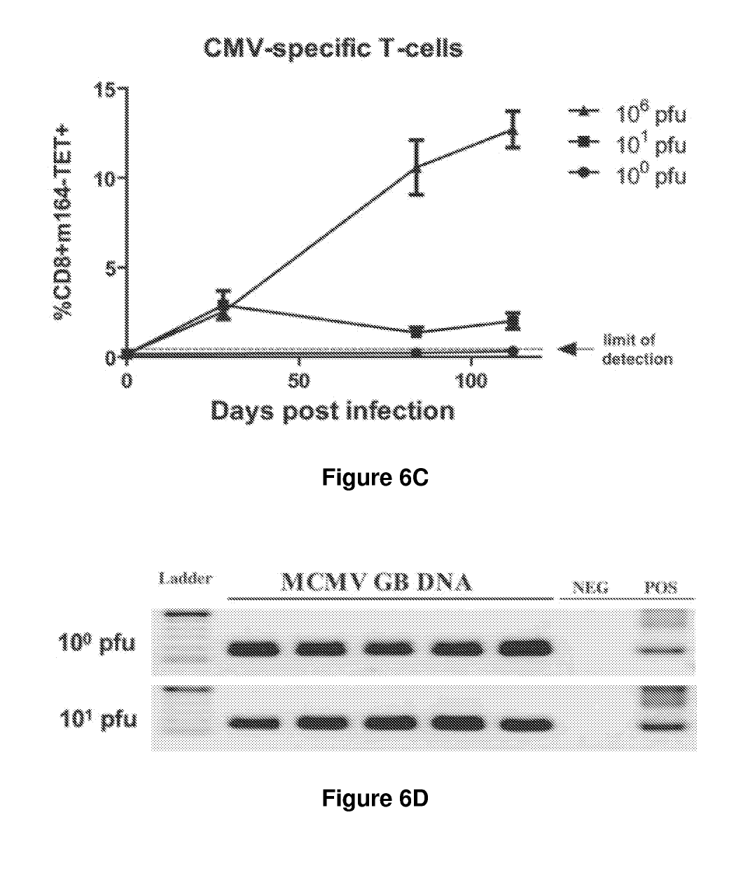

As shown in FIG. 6A, MCMV antibody responses for 10⁰ pfu were below the detection limit even 16 weeks after infection. Mice infected with 10¹ pfu had antibody levels at the detection limits 4 weeks after infection, and these became and remained just above the detection limits after 16 weeks. In contrast there are robust antibody responses induced by 10⁶ pfu infections.

For some viruses that T-cell responses might be a more sensitive indicator of infection; therefore, the inventor also measured MCMV-specific T-cell responses to immunodominant epitope MCMV-m164.

As shown in FIG. 6B and FIG. 6C, this immunodominant peptide induces “anflationary” T-memory response to 10⁶ pfu MCMV by 16 weeks after infection that makes it particularly well suited for these studies. In contrast MCMV-specific T-cell responses to 10⁰ pfu are not different from naive mice even 16 weeks after infection. Infection with 10¹ pfu induces a modest but measurable MCMV-specific T-cell response. Identical results were observed for MCMV—pp 8 9 (data not shown). Most importantly, viral DNA was detectable in lungs (FIG. 6D) and salivary gland (not shown).

Discussion

This example shows that vivarium-housed mice can develop occult MCMV that may be missed by currently available commercial serologic testing despite being in barrier housing. These MCMV-infected mice do not develop detectable MCMV-reactive antibody, but they do have MCMV DNA in their tissues. Their MCMV DNA concentrations are very low, requiring very sensitive nested PCR for detection, suggesting that these occult infections are very low titer. Indeed, we confirm that very low titer infections can occur without detectable antibody or T-cell responses. While the vehicle of these occult infections remains unknown, the inventor herein now believes that the association of occult MCMV with graft losses is very important to those involved in murine allograft transplantation or immunology studies.

Similar to the observations in mice, some seronegative human patients have been found to harbor human HCMV DNA when tested using highly sensitive PCR-based assays. The present example study directly shows directly that very low titer MCMV infections (10⁰ pfu) do not elicit antibody responses detectable by commercially available serologic methods despite detectable DNA in tissues. Viral load in end organs after MCMV infection has been shown to correlate directly with the infecting inoculum. The viral DNA correlates with viral load, and that after high titer infections MCMV can be detected by a single round of quantitative PCR. Detection of all occult infections required a second nested-PCR reaction precluding quantitation, and these findings are consistent with extremely low DNA quantities. Likewise after deliberate low titer infections, CMV DNA was not detectable by quantitative PCR (not shown). Altogether these data show that occult MCMV infections are very low titer. Importantly, for those utilizing murine transplant models, development of occult MCMV infections might elude detection by commercially available serologic methods, and suspicion of such infections should prompt evaluation of available tissues by nested PCR.

CMV has a well-established association with graft loss following human transplantation. Also, latent MCMV can influence allograft acceptance for murine cardiac transplantation, and accelerated cardiac allograft rejection in mice can follow acute infection. As such, the inventor evaluated tissues from “naïve” mice with unexpected graft losses.

The kinetic analysis shows that vivarium-housed non-immunosuppressed mice can acquire occult MCMV between 30 and 60 days after arrival. This easily overlaps the early time period after mice typically receive cardiac allografts, a precarious period that may be particularly vulnerable to viral infection.

While the inventor considered the possibility that occult MCMV might actually represent false positives from DNA contamination, several observations suggest otherwise. First, mice arriving from the vendor were all MCMV negative, as was another cohort of MCMV naïve mice housed in another facility. Second, the inventor's technique controls were consistently negative. Finally, sequencing shows that the viruses isolated from chow and wild mice were significantly different from the occult MCMV strain isolated from vivarium-housed mice. If specimen contamination was occurring during processing, then all of the sequences to be the same.

Also, it is to be noted that, although we experimentally study MCMV infection and reactivation in mice using a high titer (10⁶ pfu) infection model, we maintain these experimentally infected mice within an animal facility geographically separate from our transplant mouse colonies to minimize risk of inadvertent transmission.

The sequencing results do not support chow or wild mouse contact as a route of transmission. In fact, the results show that vivarium-housed mice are somehow being infected with the laboratory MCMV strain. Possibilities include improper animal handling, inadequate disinfection of caging/supplies, or viral contamination of bedding, water, or personnel protective attire.

Nevertheless, these data confirm that hosts infected with low titer CM V may not develop appreciable CMV-specific antibody or T-cell immune responses. Mice housed in a barrier vivarium for more than 30-60 days can develop occult MCMV that may be missed by currently available commercial serologic testing. It is worrisome that these occult infections may significantly influence murine models of transplantation and completely escape notice.

Thus, the sensitive method now described herein is especially useful to evaluate tissues acquired from mice for presence of MCMV for varied usages in immunocompromised specimens.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to the particular embodiment disclosed herein contemplated for carrying out this invention, but that the invention will include all embodiments falling within the scope of the claims.

1. A PCR set comprising a primer set including a primer having a nucleotide sequence as set forth in SEQ ID NO: 1, or an operable fragment thereof, and a primer having a nucleotide sequence as set forth in SEQ ID NO:2, or an operable fragment thereof,
wherein the primer set specifically amplifies a target region of murine cytomegalovirus (MCMV) in a polymerase chain reaction (PCR).

2. A PCR set comprising a nested primer set including a primer having a nucleotide sequence as set forth in SEQ ID NO: 3, or an operable fragment thereof, and a primer having a nucleotide sequence as set forth in SEQ ID NO:4, or an operable fragment thereof,
wherein the nested primer set specifically amplifies a target region of murine cytomegalovirus (MCMV) in a polymerase chain reaction (PCR).

3. A PCR set comprising a primer set including a primer having a nucleotide sequence as set forth in SEQ ID NO: 1, or an operable fragment thereof, and a primer having a nucleotide sequence as set forth in SEQ ID NO:2, or an operable fragment thereof, and, a nested primer set including a primer having a nucleotide sequence as set forth in SEQ ID NO: 3, or an operable fragment thereof, and a primer having a nucleotide sequence as set forth in SEQ ID NO:4, or an operable fragment thereof;
wherein the nested primer set specifically amplifies a target region of murine cytomegalovirus (MCMV) in a polymerase chain reaction (PCR).

4. (canceled)

5. (canceled)

6. The method of claim 21, wherein the PCR is performed under conditions of initial denaturation 4 min at 94° C., 35 cycles-denaturation 30 s at 94° C., annealing 30 s at 53° C., elongation 30 s at 72° C., followed by final elongation 7 min at 72° C., then holding at 4° C.

7. (canceled)

8. (canceled)

9. (canceled)

10. (canceled)

11. (canceled)

12. The method of claim 21, wherein the sample comprises one or more of: blood, serum, plasma, sputum, urine, stool, skin, cerebrospinal fluid, saliva, gastric secretions, semen, seminal fluid, breastmilk, tears, oropharyngeal swabs, nasopharyngeal swabs, throat swabs, nasal aspirates, nasal wash, fluids collected from the ear, eye, mouth, respiratory airways, spinal tissue or fluid, cerebral fluid, trigeminal ganglion sample, a sacral ganglion sample, adipose tissue, lymphoid tissue, placental tissue, upper reproductive tract tissue, gastrointestinal tract tissue, male genital tissue and fetal central nervous system tissue.

13. The method of claim 12, wherein the sample is from a mouse.

14. The method of claim 12, wherein the sample is from a human.

15. (canceled)

16. (canceled)

17. (canceled)

18. (canceled)

19. An oligonucleotide primer comprising the sequence (SEQ ID NO:1, 2, 3 or 4) or a portion of SEQ ID NO: 1, 2, 3 or 4,
wherein the portion of the primer comprises at least one unique nucleotide as identified as a mismatch between strains of murine cytomegalovirus (MCMV) from other herpes viruses, and distinguishes from other strains of herpes virus.

20. A kit for detecting murine cytomegalovirus (MCMV), comprising (a) a pair of flanking primers comprised of SEQ ID NO: land SEQ ID NO:2 and (b) a pair of nested primers comprised of SEQ ID NO:3 and SEQ ID NO: 4.

21. A method for detecting murine cytomegalovirus (MCMV), comprising:
a) providing an amplified cDNA of a sample though RT-PCR using a pair of primers comprised of SEQ ID NO:1 and SEQ ID NO:2;
b) discriminating the amplified cDNA of step (a) though nested PCR using a pair of SNP primers consisting comprised of SEQ ID NO:3 and SEQ ID NO:4; and
c) identifying the amplified cDNA of step (b) and detecting MCMV, if present.

22. A method of detecting and identifying mammalian Herpesvirus of the type Cyclomegalovirus in a sample, comprising the steps of:
a) applying onto a supporting substrate consensus DNA polymerase gene standards corresponding to respective mammalian Cyclomegalovirus;
b) isolating DNA from the sample of bodily fluid;
c) amplifying the isolated DNA by a round of PCR using designed flanking primers of the sequences:
Flanking Primer—left: ctg ggc gag aac aac gag at [SEQ ID NO:1] and
Flanking Primer—right: cgc agc tct ccc ttc gag ta, [SEQ ID NO:2], thereby generating PCR products;
d) hybridizing the PCR products of step c) to the supporting substrate; and
e) detecting bound PCR products of step d):
wherein a presence of bound PCR product indicates the presence of the mammalian Herpesvirus in the sample.

23. (canceled)

24. The method of claim 22, wherein the sample comprises one or more of: saliva, saliva gland extract, urine, bladder extract, kidney extract, blood, lung extract, tears, semen or breast milk.

25. The method of claim 22, wherein the mammal is a mouse, rat, guinea pig, swine, pig, hog or boar.

26. The method of claim 22, wherein the mammal comprises an immunocompromised mammal.

27. The method of claim 26, wherein the immunocompromised mammal is selected from mammals receiving allograft; mammals infected with AIDS or AIDS like virus; mammals having leukemia; mammal fetus; newborn mammals.

28. A kit for detecting and identifying mammalian Herpesvirus in a sample, comprising:
1) consensus DNA polymerase gene standards corresponding to mammalian Cytomegalovirus;
2) a supporting substrate to which the consensus DNA standards can be attached;
3) Flanking Primer—left: ctg ggc gag aac aac gag at [SEQ ID NO:1]; and
4) Flanking Primer—right: cgc agc tct ccc ttc gag ta, [SEQ ID NO:2].

29. The kit of claim 28, wherein the kit contains materials for a second PCR cycle, comprising:
Nested Primer—left: gag aac tgc gac acg aac ag [SEQ ID NO:3]; and
Nested Primer—right: agc acc ttg aag tcg gtg tt [SEQ ID NO:4].

30. The kit of claim 28, wherein the mammalian Herpesvirus is Cytomegalovirus.

31. (canceled)

32. A method of avoiding Herpes related complication in surgical procedure on mammals, comprising: detecting and identifying mammalian Herpesvirus in a sample taken from the mammal using the method of claim 22.

33. (canceled)

34. The method of claim 32, wherein the surgery is tissue transplantation.

35. The method of claim 32, wherein the surgery is on an immunocompromised mammal, mammals infected with AIDS or AIDS like virus; having leukemia; mammal fetus; or newborn mammal.

36. The method of claim 32, wherein the sample is one or more of: saliva, saliva gland extract, urine, bladder extract, kidney extract, blood, lung extract, tears, semen or breast milk.

37. (canceled)

38. (canceled)

39. A method according to claim 32, wherein the sample comprises a cell culture medium containing mammalian cultured cells.

40. A method according to claim 37, wherein the sample comprises cells obtained from tissue harvested from mammals.

41. A method according to claim 32, wherein the sample comprises embryonic cells.

42. A method for differentiating an infectious strain from a vaccine strain comprising:
a) providing an amplified cDNA of a sample through RT-PCR by a pair of primers having a nucleotide sequence as set forth in SEQ ID NO:1 and SEQ ID NO:2, wherein the pair of primers share the same template from the infectious and vaccine strains;
b) discriminating the amplified cDNA of step (a) through nested PCR by a pair of nested primers having a nucleotide sequence as set forth in SEQ ID NO:3 and SEQ ID NO:4, which creates incomplete complementarity to the amplified cDNA of step (a); and
c) identifying the amplified cDNA of step (b) and detecting MCMV, if present.

43. An isolated nucleic acid comprising a nucleotide sequence as set forth in SEQ ID NO:1.

44. An isolated nucleic acid comprising a nucleotide sequence as set forth in SEQ ID NO:2.

45. An isolated nucleic acid comprising a nucleotide sequence as set forth in SEQ ID NO:3.

46. An isolated nucleic acid comprising a nucleotide sequence as set forth in SEQ ID NO:4.


SEQUENCE LISTING (23 sequences)

SEQ ID NO: 1 - DNA, 20 bp, Artificial Sequence
  Description of Artificial Sequence Synthetic primer
ctgggcgagaacaacgagat

SEQ ID NO: 2 - DNA, 20 bp, Artificial Sequence
  Description of Artificial Sequence Synthetic primer
cgcagctctcccttcgagta

SEQ ID NO: 3 - DNA, 20 bp, Artificial Sequence
  Description of Artificial Sequence Synthetic primer
gagaactgcgacacgaacag

SEQ ID NO: 4 - DNA, 20 bp, Artificial Sequence
  Description of Artificial Sequence Synthetic primer
agcaccttgaagtcggtgtt

SEQ ID NO: 5 - DNA, 22 bp, Artificial Sequence
  Description of Artificial Sequence Synthetic primer
tagccaatgatatcttcgagcg

SEQ ID NO: 6 - DNA, 25 bp, Artificial Sequence
  Description of Artificial Sequence Synthetic primer
atctggtgctcctcagatcagctaa

SEQ ID NO: 7 - DNA, 22 bp, Artificial Sequence
  Description of Artificial Sequence Synthetic primer
gggaccctactccgacgacgtg

SEQ ID NO: 8 - DNA, 22 bp, Artificial Sequence
  Description of Artificial Sequence Synthetic primer
gctctgctcttcgatcggtagg

SEQ ID NO: 9 - DNA, 22 bp, Artificial Sequence
  Description of Artificial Sequence Synthetic primer
acaacgcaagatgatatacggc

SEQ ID NO: 10 - DNA, 23 bp, Artificial Sequence
  Description of Artificial Sequence Synthetic primer
actaccacatgtgtggatacgct

SEQ ID NO: 11 - DNA, 22 bp, Artificial Sequence
  Description of Artificial Sequence Synthetic primer
gacctcaagaacaccgtctacg

SEQ ID NO: 12 - DNA, 21 bp, Artificial Sequence
  Description of Artificial Sequence Synthetic primer
caggaagcgcgccaagggatc

SEQ ID NO: 13 - DNA, 20 bp, Artificial Sequence
  Description of Artificial Sequence Synthetic primer
tggattccatcaatgcaaaa

SEQ ID NO: 14 - DNA, 20 bp, Artificial Sequence
  Description of Artificial Sequence Synthetic primer
gaggagctccatcactcgtc

SEQ ID NO: 15 - protein, 9 aa, Murine cytomegalovirus
YPHFMPTNL

SEQ ID NO: 16 - protein, 9 aa, Murine cytomegalovirus
AGPPRYSRI

SEQ ID NO: 17 - DNA, 39 bp, Murine cytomegalovirus
tctgctgccatactgccagctagacctggcatccaggaa

SEQ ID NO: 18 - DNA, 38 bp, Murine cytomegalovirus
tctgctgccatactgccagctgacctggcatccaggaa

SEQ ID NO: 19 - DNA, 40 bp, Murine cytomegalovirus
tctgctgccatactgccagctgagacctggcatccaggaa

SEQ ID NO: 20 - DNA, 40 bp, Murine cytomegalovirus; identical to SEQ ID NO: 19

SEQ ID NO: 21 - DNA, 37 bp, Murine cytomegalovirus
tctgctgccatactgccagctgcctggcatccaggaa

SEQ ID NO: 22 - DNA, 37 bp, Murine cytomegalovirus; identical to SEQ ID NO: 21

SEQ ID NO: 23 - DNA, 37 bp, Murine cytomegalovirus; identical to SEQ ID NO: 21